(19) United States
(12) Patent Application Publication
(10) Pub. No.: US 2004/0077888 A1
(43) Pub. Date: Apr. 22, 2004

(54) Polyamine-mediated transfection

(75) Inventors: Edmund J. Niedzinski, Vacaville, CA (US); Michael H. Nantz, Davis, CA (US); Michael Bennett, El Sobrante, CA (US); Mike E. Lizarzaburu, San Mateo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: The Regents of the University of California, Oakland, CA (US); Genteric, Inc., Alameda, CA (US)

(21) Appl. No.: 10/448,641

(22) Filed: May 30, 2003

Related U.S. Application Data
(60) Provisional application No. 60/385,234, filed on May 31, 2002.
(60) Provisional application No. 60/384,514, filed on May 31, 2002.

(51) Int. Cl. C07J 9/00

(52) U.S. Cl. 552/544; 560/149

(57) ABSTRACT

The present invention provides cationic lipids and methods that facilitate nucleic acid delivery and which also yield biologically benign metabolites following transfection. The compounds are lipidic polyamines that use a pentaerythritol scaffold to link different hydrophobic and DNA-binding domains.

### CROSS-REFERENCE TO RELATED APPLICATIONS

This application claims priority to U.S. Provisional Patent Application Nos. 60/385,234 and 60/384,514, both filed on May 31, 2002, the disclosures of which are hereby incorporated by reference in their entireties for all purposes.

### BACKGROUND OF THE INVENTION

The development of new cationic lipids as vehicles for the intracellular delivery of gene pharmaceuticals continues to be a promising field of research (see, Miller, A. D., Angew. Chem. Int. Ed., 37:1768-1785 (1998)). Since Felgner's first report (see, Feigner et al., Proc. Natl. Acad. Sci. USA., 84:7413-7417 (1987)) of cationic lipid-mediated DNA delivery, dramatic improvements in lipid-based gene delivery systems have led to numerous clinical applications (see, Martin, P., J. Gene Med., 3:91-92 (2001)). Despite recent successes in non-viral gene therapy, cytotoxicity and low transfection activity continue to plague most lipid-based gene delivery systems (see, Byk et al., European Opin. Ther. Patents, 8:1125-1141 (1998)). Overcoming these obstacles is crucial for successful application of this technology, particularly in cases that involve repeat dosing. One particular approach to ameliorate the effects of lipid-associated cytotoxicity is the design and synthesis of degradable cationic lipids. Lipids may be engineered to degrade in response to a number of physiological conditions, such as changes in pH (see, Zhu et al, J. Am. Chem. Soc., 122:2645-2646 (2000); Gerasimov et al., Biochim. Biophys. Acta., 1324:200-214 (1997)) or enzymatic activity (see, Litzinger et al., Biochim. Biophys. Acta., 1113:201-227 (1992)). A cationic lipid that slowly degrades to non-toxic metabolites after facilitating the cellular incorporation of DNA would avoid the potential side effects that might result from the cumulative concentration of intracellular cationic lipids. The development of cationic lipids that both facilitate DNA delivery and also yield biologically benign metabolites following DNA transfection would thus be advantageous (see, Byk et al., J. Med. Chem., 43:4377-4387 (2000); Tang et al., Biochem. Biophys. Res. Commun., 242:141-145, (1998)).

Previous research has explored the relationship between the chemical structure of cationic lipids and the influences on non-viral gene transfection (see, Wu et al., Bioconjugate Chem., 12:251-257 (2001); Bennett et al., J. Med. Chem., 40:4069-4078 (1997)). In one such study, pentaerythritol derivatives containing α-ammonium ester headgroups exhibited low cytotoxicity in transfection experiments (see, Aberle et al., Biochemistry, 37:6533-6540). As postulated, the metabolism of these compounds was accelerated by facile hydrolysis of the α-ammonium esters, giving rise to non-toxic metabolites.

In view of the foregoing, what is needed in the art is the development of cationic lipids that both facilitate DNA delivery and also yield biologically benign metabolites following DNA transfection. The present invention satisfies these and other needs.

### SUMMARY OF THE INVENTION

The present invention provides cationic lipids and methods that facilitate nucleic acid delivery and which also yield biologically benign metabolites following transfection. The compounds are lipidic polyamines that use a pentaerythritol scaffold to link different hydrophobic and DNA-binding domains.

In one aspect, the present invention provides a method for designing a lipoplex targeted for a specific cell, comprising: providing a lipoplex comprising a nucleic acid and a pentaerythritol polyamine (PEP) compound; and varying the structural features of the pentaerythritol polyamine (PEP) compound having a hydrophobic domain and a nucleic acid binding domain to impart cell selectivity, thereby designing a lipoplex targeted for the specific cell.

In certain preferred aspects, the PEP compound has Formula I:

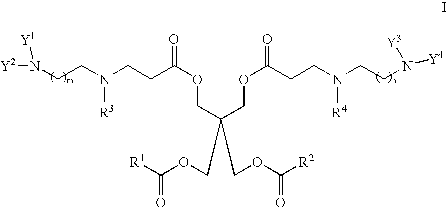

In Formula I, R¹ and R² are each members independently selected from optionally substituted C₈-C₂₄ alkyl, optionally substituted C₈-C₂₄ alkenyl, and cholesteryl (C₂₇—H₄₅O forming for example, a carbonate). In Formula I, R³ and R⁴ are each members independently selected from hydrogen, and optionally substituted C₁-C₄ alkyl. In Formula I, Y¹, Y², Y³ and Y⁴ are each members independently selected from hydrogen and optionally substituted C₁-C₆ alkyl. In Formula I, m and n are integers independently selected from about 1 to about 4, provided that m is not equal to n unless m and n are equal to 4; or a pharmaceutically acceptable salt thereof.

In another aspect, the present invention provides a compound of Formula I:

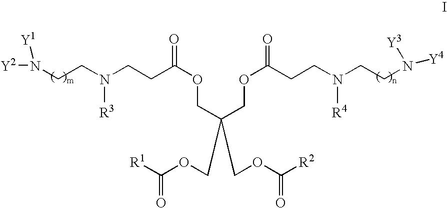

In Formula I, R¹ and R² are each members independently selected optionally substituted C₈-C₂₄ alkyl, optionally substituted C₈-C₂₄ alkenyl, and cholesteryl. In Formula I, R³ and R⁴ are each members independently selected from hydrogen, and optionally substituted C₁-C₄ alkyl. In Formula I, Y¹, Y², Y³ and Y⁴ are each members independently selected from hydrogen and optionally substituted C₁-C₆ alkyl. In Formula I, m and n are integers independently selected from about 1 to about 4, provided that m is not equal to n unless m and n are equal to 4; or a pharmaceutically acceptable salt thereof.

In yet another aspect, this invention relates to a method for transfecting a nucleic acid into a cell. In this method, the cell is contacted with a lipid-nucleic acid complex, or liposome, the lipid portion of which contains a PEP compound of Formula I. Using standard techniques, the lipids of Formula I can facilitate the transfection of nucleic acids into cells, in vivo and in vitro, with high efficiency.

In still yet another aspect, this invention relates to a pharmaceutical composition or other drug delivery composition for administering a nucleic acid particle to a cell. This composition includes a lipid-nucleic acid complex, the lipid portion of which contains a PEP compound of Formula I, and a pharmaceutically acceptable carrier.

In yet another aspect of the invention provides methods of treating diseases arising from infection by a pathogen or from an endogenous DNA deficiency. These methods involve administering a liposome-nucleic acid aggregate and/or liposome-drug aggregate solution to a mammal suffering from a pathogenic infection or DNA deficiency.

These and other advantages, objects and embodiments will become more apparent when read with the accompanying detailed description and drawings which follow.

### DESCRIPTION OF THE DRAWINGS

FIGS. 1A-C illustrate the effect of pH on DNA condensation by polyamine Panel A compound 8, Panel B compound 9, and Panel C compound 10 and cholesterol were added to 0.5 μg pCMV-luc DNA that was pre-incubated with ethidium bromide. The lipoplexes were then treated with either water (diamond), 10 mM Tris pH=7 (square), 10 mM Tris pH=8 (triangle), or 10 mM Tris pH=9 (open square). The resulting fluorescence was measured at 250 nm excitation and 610 nm emission. Each data point reflects the mean value from three samples, and the standard deviation from the mean.

FIG. 2 illustrates CHO cell transfection using polyamines 5-10. Polyamine formulations (compounds depicted as alternative black and white bars) containing equimolar DOPE (Condition A) or cholesterol (Condition B) were complexed with luciferase pDNA at the indicated N:P ratios and used to deliver 1 μg DNA/well. Luciferase activity in the cell lysates was determined 24 hours after the transfection. Each data point reflects the mean value from three transfections, and the standard deviation from the mean.

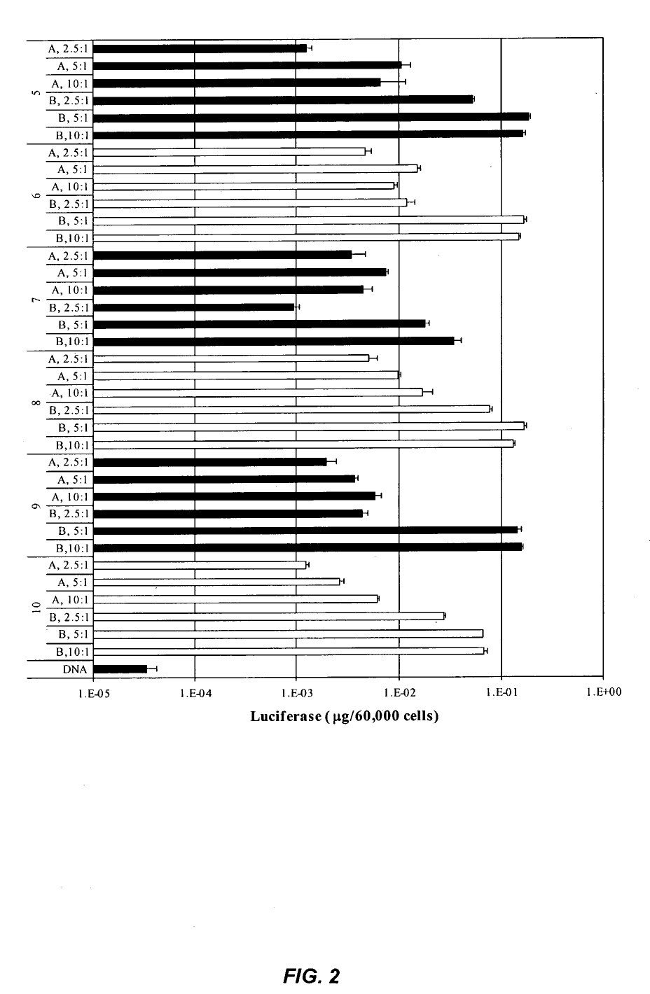

FIG. 3 illustrates the analysis of transfection activity in CHO cells using a luciferase assay. Polyamine 5-10 formulations containing equimolar cholesterol were complexed with luciferase plasmid at a 5:1 N:P ratio and used to deliver 1 μg DNA/well. LipofectAMINE PLUS (LFP) and TransFast (TF) were formulated at N:P ratios of 5:1 and 3:1 respectively and used to deliver 1 μg DNA/well. Luciferase activity in the cell lysates was determined 24 hours after the transfection. Each data point reflects the mean value from three transfections, and the standard deviation from the mean.

FIG. 4 illustrates the analysis of transfection activity in NIH-3T3 cells using a luciferase assay. Polyamine 5-10 formulations containing equimolar cholesterol were complexed with luciferase plasmid at a 10:1 N:P ratio and used to deliver 1 μg DNA/well. LipofectAMINE PLUS (LFP) and TransFast (TF) were formulated at N:P ratios of 5:1 and 3:1 respectively and used to deliver 1 μg DNA/well. Luciferase activity in the cell lysates was determined 24 hours after the transfection. Each data point reflects the mean value from three transfections, and the standard deviation from the mean.

FIG. 5 illustrates the analysis of transfection activity and cytotoxicity in CHO cells as measured by flow cytometry. Polyamine 5-10 formulations containing equimolar cholesterol were complexed with GFP plasmid at a 5:1 N:P ratio and used to deliver 1 μg DNA/well. Twenty-four hours after transfection, cells were analyzed for GFP fluorescence to determine the transfection activity and Annexin V binding to determine cytotoxicity. The black bars represent the percentage of the cell population that was GFP positive and Annexin V negative. The hashed bars represent percentage of the cell population that was GFP positive and Annexin V positive. The white bars represent the percentage of the cell population that was GFP negative and Annexin V positive.

FIG. 6 illustrates the analysis of transfection activity and cytotoxicity in NIH-3T3 cells as measured by flow cytometry. Polyamine 5-10 formulations containing equimolar cholesterol were complexed with GFP plasmid at a 10:1 N:P ratio and used to deliver 1 μg DNA/well. Twenty-four hours after transfection, cells were analyzed for GFP fluorescence to determine the transfection activity and Annexin V binding to determine cytotoxicity. The black bars represent the percentage of the cell population that was GFP positive and Annexin V negative. The hashed bars represent percentage of the cell population that was GFP positive and Annexin V positive. The white bars represent the percentage of the cell population that was GFP negative and Annexin V positive. The remaining percentage of the cell population that was GFP negative and Annexin V negative are not represented.

### DETAILED DESCRIPTION OF THE INVENTION AND PREFERRED EMBODIMENTS

A. Definitions

As used herein, the term “alkyl” denotes branched or unbranched hydrocarbon chains, preferably having about 1 to about 24 carbons. These groups can be optionally substituted with one or more functional groups which are attached commonly to such chains, such as, hydroxyl, bromo, fluoro, chloro, iodo, mercapto or thio, cyano, alkylthio, heterocyclyl, aryl, heteroaryl, carboxyl, carbalkoyl, alkyl, alkenyl, nitro, amino, alkoxyl, amido, and the like to form alkyl groups such as trifluoro methyl, 3-hydroxyhexyl, 2-carboxypropyl, 2-fluoroethyl, carboxymethyl, cyanobutyl and the like.

The term “alkenyl” denotes branched or unbranched hydrocarbon chains containing one or more carbon-carbon double bonds, preferably having about 8 to about 24 carbons.

The term “nucleic acid” refers to a polymer containing at least two nucleotides. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleotides are the monomeric units of nucleic acid polymers. A “polynucleotide” is distinguished here from an “oligonucleotide” by containing more than 80 monomeric units; oligonucleotides contain from 2 to 80 nucleotides. The term nucleic acid includes deoxyribonucleic acid (DNA) and ribonucleic acid (RNA). The term encompasses sequences that include any of the known base analogs of DNA and RNA.

DNA may be in the form of anti-sense, plasmid DNA, parts of a plasmid DNA, product of a polymerase chain reaction (PCR), vectors (P1, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), anti-sense RNA, ribozymes, chimeric sequences, or derivatives of these groups.

“Antisense” is a polynucleotide that interferes with the function of DNA and/or RNA. This may result in suppression of expression. Natural nucleic acids have a phosphate backbone, artificial nucleic acids may contain other types of backbones and bases. These include PNAs (peptide nucleic acids), phosphothionates, and other variants of the phosphate backbone of native nucleic acids. In addition, DNA and RNA may be single, double, triple, or quadruple stranded.

The term “recombinant DNA molecule” as used herein refers to a DNA molecule that is comprised of segments of DNA joined together by means of molecular biological techniques. “Expression cassette” refers to a natural or recombinantly produced polynucleotide molecule that is capable of expressing protein(s). A DNA expression cassette typically includes a promoter (allowing transcription initiation), and a sequence encoding one or more proteins. Optionally, the expression cassette may include trancriptional enhancers, noncoding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences.

The term “gene” refers to a nucleic acid (e.g., DNA) sequence that comprises coding sequences necessary for the production of a polypeptide or precursor (e.g., myosin heavy chain). The polypeptide can be encoded by a full length coding sequence or by any portion of the coding sequence so long as the desired activity or functional properties (e.g., enzymatic activity, ligand binding, signal transduction, and the like) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ nontranslated sequences. The term “gene” encompasses both cDNA and genomic forms of a gene. A genomic form or clone of a gene contains the coding region interrupted with noncoding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Upregulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

B. Compounds

In one embodiment, the present invention provides polyamines useful in the transfection of nucleic acid. The polyamines possess a pentaerythritol structure with a hydrophobic domain and a nucleic acid-binding domain. As described in more detail below, the transfection-active headgroup and sidechain domains can be attached using straightforward chemistry.

In one embodiment, the present invention provides a compound of Formula I:

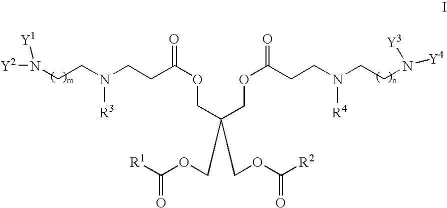

wherein R¹, R², R³, R⁴ Y¹, Y², Y³, Y⁴, m, and n have previously been defined. In certain embodiments, R¹ and R² are preferably C₈-C₂₀ alkyl or C₈-C₂₀ alkenyl. The substituent C₈-C₂₀ alkyl includes, for example, C₈ alkyl, C₉ alkyl, C₁₀ alkyl, C₁₁ alkyl, C₁₂ alkyl, C₁₃ alkyl, C₁₄ alkyl, C₁₅ alkyl, C₁₆ alkyl, C₁₇ alkyl, C₁₈alkyl, C₁₉ alkyl, and C₂₀ alkyl. The substituent C₈-C₂₀ alkenyl includes, for example, C₉ alkenyl, C₉ alkenyl, C₁₀alkenyl, C₁₁ alkenyl, C₁₂ alkenyl, C₁₃ alkenyl, C₁₄ alkenyl, C₁₅ alkenyl, C₁₆ alkenyl, C₁₇ alkenyl, C₁₈ alkenyl, C₁₉ alkenyl, and C₂₀ alkenyl. In certain other aspects, Y¹, Y², Y³, and Y⁴ are independently hydrogen, methyl, ethyl, propyl and butyl, and m and n are integers independently selected from about 1-2.

In one preferred aspect, R¹ and R² are each independently selected from C₁₃ alkyl (forming for example, myristoyl), C₁₇ alkenyl (forming for example, oleoyl), C₁₁ alkyl (forming for example, lauroyl), C₁₇ alkyl (forming for example stearoyl) and C₁₅ alkyl (forming for example, palmitoyl) and Y¹, Y², Y³ and Y⁴ are each members independently selected from hydrogen and methyl; and m and n are integers independently selected from about 1-2. In other aspects, R¹ and R² are both C₁₇ alkenyl (forming for example, oleoyl), or both C₁₃ alkyl (forming for example, myristoyl).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent.

Examples of pharmaceutically acceptable acid addition salts include, but are not limited to, those derived from inorganic acids like hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Certain specific compounds of the present invention contain both basic and acidic functionalities that allow the compounds to be converted into either base or acid addition salts.

In certain embodiments, compounds of Formula I have at least one nitrogen that is quaternized with the addition of an alkyl group, such as a methyl group, to the at least one nitrogen, to form for example, a tetramethyl ammonium chloride salt. In certain aspects, at least two nitrogens are quaternized. In other aspects, at least three nitrogens are quaternized, and in some embodiments, all the nitrogens are quaternized with the addition of alkyl group, to form an ammonium salt. As such, in certain aspects, the salts of the present invention exist as quaternized nitrogen salts including, but not limited to, quaternary ammonium chloride, a quaternary ammonium iodide, a quaternary ammonium fluoride, a quaternary ammonium bromide, a quaternary ammonium oxyanion and a combination thereof. As used herein, the term a quaternary ammonium salt include 1, 2, or 3 alkyl groups on the nitrogen atom. When more than one nitrogen exists in the PEP molecule, any or all nitrogens can be quaternized, that is 1, 2, 3 or more nitrogens can be quaternized.

In certain aspects, the compounds of the present invention can be synthesized using a procedure outlined in the scheme below. As shown therein, a pentaerythritol structure in “step a” is esterified by addition of acryloyl chloride to yield a bisacrylate. Thereafter, in “step b,” a diamine such as 2-(N,N-dimethyl)ethanamine, 3-(N,N-dimethylamino)propanamine, 4-(N,N-dimethylamino)butanamine or the like, is used to generate the compounds of the present invention.

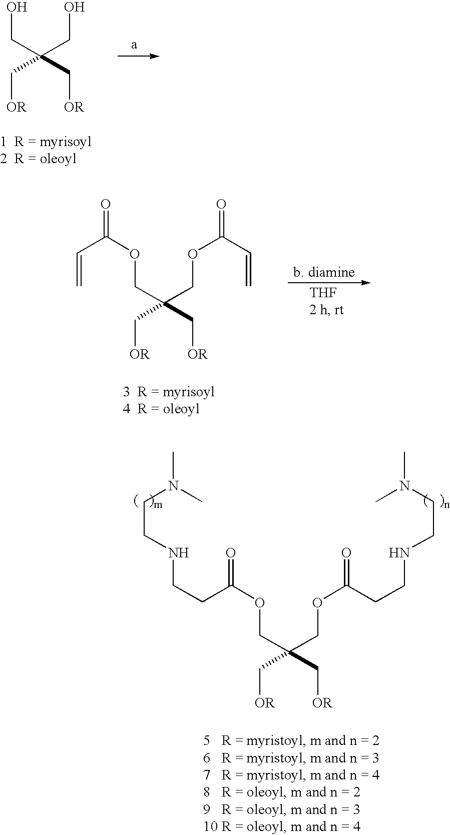

In an illustrative embodiment, dioleoyl (C_{18:1}) and dimyristoyl (C_{14:0}) analogs of pentaerythritol are treated with acryloyl chloride under standard acylation conditions to obtain the respective diacrylate esters 3 and 4 (Scheme 1). Thereafter, the polyamine headgroups are incorporated by conjugate addition of three different (N,N-dimethylamino)alkanamines to give the polyamines (structures 5-10 as shown in Scheme 1). After removal of excess diamine by aqueous extraction, the polyamine lipids of the present invention are obtained as homogenous compounds.

The present invention demonstrates that pentaerythritol polyamine (PEP) compounds function as versatile transfection lipids. The synthetic versatility of this core structure allows for the introduction of structurally diverse hydrophobic and DNA binding domains via ester bond formation. Advantageously, the low cytotoxicity of the PEP compounds, due in part to their susceptibility to ester hydrolysis under physiological conditions, greatly facilitates their metabolism. The rapid clearance of a cationic lipid construct is an important factor in maintaining normal cellular function as the inadvertent binding of cationic lipids to protein kinase C (and other essential cellular enzymes) is regarded as an origin of cytotoxicity (see, Farhood et al., Biochim. Biophys. Acta., 1111:239-246 (1992)).

In certain aspects, the present invention provides a class of transfection agents by attachment of a multivalent DNA-binding domain. The pentaerythritol polyamine (PEP) compounds facilitate intracellular delivery of DNA, which readily degrade via ester hydrolysis to minimize cytotoxic side-effects.

In certain aspects, the pentaerythritol tetraesters e.g., 3 and 4, serve as versatile building blocks for the introduction of various headgroup structures by straightforward conjugate addition reactions. In certain preferred aspects, the polyamines are more active when formulated with cholesterol.

In one aspect, the PEPs condense the DNA, making it more compact and easier to deliver. The polyamines can be analyzed for their ability to bind and condense DNA by measuring the fluorescence of intercalated ethidium bromide. In one aspect, pH has an effect on DNA condensation by the polyamines.

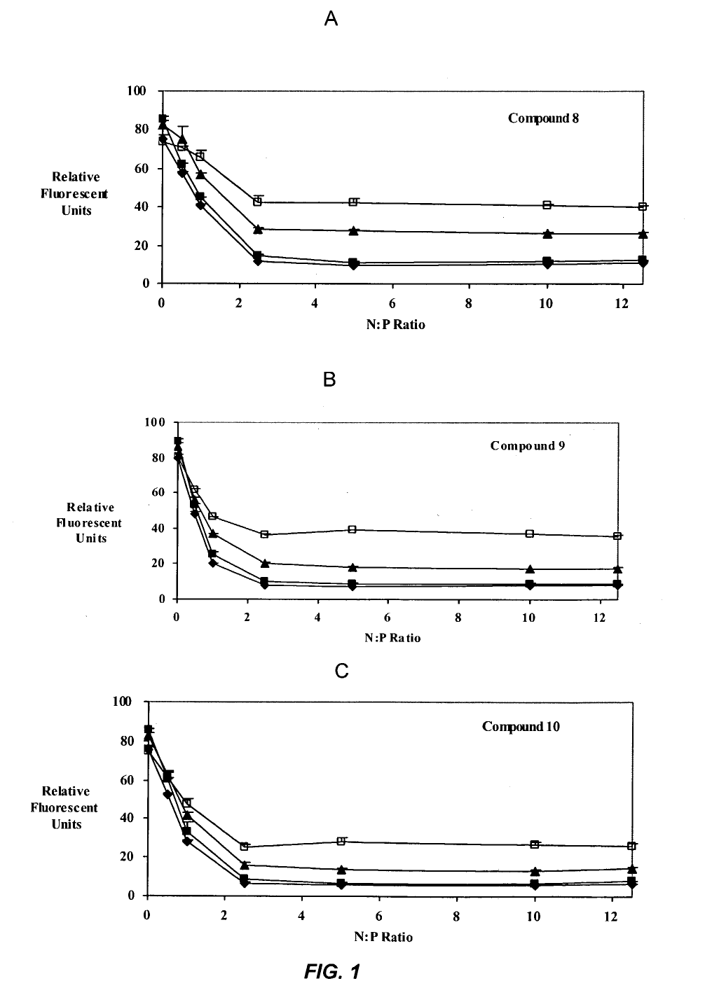

The assay set forth in FIGS. 1A-C is based on a decrease in fluorescence that occurs when ethidium bromide is displaced from DNA by adding cationic liposomes. The lipoplexes were formed by adding a DNA-ethidium bromide solution to a solution containing variable amounts of polyamine:cholesterol liposomes. The lipoplexes were then treated with an equal volume of either water or a TRIS buffered solution at various pHs. The resulting fluorescence was then measured to determine the relative concentration of intercalated ethidium bromide.

As shown therein, all three head groups of compounds 8, 9 and 10 condensed DNA with efficiency. The N/P ratios are calculated values based on the net ratio of amines in the lipid headgroup to DNA phosphate residues. In one aspect, at N/P ratios ≧2.5:1 in water and in TRIS buffer (pH=7), the relative fluorescent units remain constant, indicating that the DNA is fully and efficiently condensed. When the pH is increased, the fluorescence increases. For example, at a N/P ratio of 10:1, the fluorescence ranged from 6.06 to 10.24 relative fluorescent units (RFU) when the formulations were treated with TRIS pH=7. When the same formulations were treated with TRIS pH=9, the fluorescence increased to 26.6 to 40.8 RFU. Similar trends were observed when these polyamines were co-formulated with DOPE.

As shown in FIG. 1, the DNA is completely compacted at neutral pH. This pH dependence can be attributed to titration of the polyamine head groups. The amines in the DNA-binding domain begin to deprotonate at a higher pH. In certain aspects, the lipoplexes are formulated at an N/P ratio greater than or equal to 2.5:1.

C. Lipoplex Design

In one aspect, the present invention provides a method for designing a lipoplex targeted for a specific cell, comprising: providing a lipoplex comprising a nucleic acid and a pentaerythritol polyamine (PEP) compound; and varying the structural features of the pentaerythritol polyamine (PEP) compound having a hydrophobic domain and a nucleic acid binding domain to impart cell selectivity, thereby designing a lipoplex targeted for the specific cell.

In certain aspects, by varying the design of the PEP structural features it is possible to target specific tissue or a targeted cell. In certain aspects, the targeted cell is a tissue such as a tumor, an organ, or bone. The tumor can be a cancerous tumor.

Based upon the variability in lipid compositions and the different concentrations of membrane-associated proteins, in certain instances, each cell type presents a unique cell surface. For example, the fluidity of a cell membrane can vary with different concentrations of trans-membrane proteins. For non-viral gene therapy, this environmental variability presents an opportunity to design a transfection reagent that may favor a certain cell type.

The differences in activity that are observed for a structurally related series of cationic lipids-DNA formulation that are tested in a single cell type can be attributed to the phase transition temperature of the cationic lipid, which is a structurally dependent property. An optimal pairing of the phase transition temperature of the cationic lipid and fluidity of a cell-membrane is a parameter for finding an optimal cationic lipid formulation for the transfection of a particular cell type. Therefore, the analysis of a series of structurally related cationic lipids is required to elucidate the cationic lipid that will yield the most gene expression for each cell type.

In certain embodiments, this matching may be carried out in a purely empirical fashion. In other aspects, the matching is carried out with a combination of empirical finding and phase transition “matches.”

In certain aspects, the hydrophobic domain is varied by independently changing the chain length of the alkyl chain(s). For example, the alkyl or alkenyl chain can be varied by greater or lesser number of carbon atoms, substituting the chain, or increasing sites of unstaturation. In other aspects, the nucleic acid domain is varied by changing the substituent at the nitrogen atoms, counter ions and salts. Moreover, the carbon linker(s) can be varied or independently varied.

Additional structural features which can be varied in order to impart cell selectivity include for example, varying the mole ratio of amines in the PEP head group to nucleic acid phosphates (N/P). In one aspect, luciferase transfection revealed that the polyamines were most active when combined with pDNA at an N/P ratio≧5:1 in CHO cells and 10:1 in NIH 3T3 cells. Preferably, this ratio is greater than about 2.5:1. In another preferred aspect, the mole ratio of amines in the PEP head group to nucleic acid phosphates (N/P) is about 2.5:1 to about 10:1.

In one embodiment, the spacer group dictated by “m” and “n” will determine cell selectivity. For example, in compounds having similar hydrophobic domains, polyamines containing C₃-spacers had superior activity in CHO cells and C₂-spacers had superior activity in NIH-3T3 cells.

In another embodiment, polyamines were more active in NIH 3T3 cells when co-formulated with cholesterol rather than DOPE.

In certain other embodiments, specific targeting moieties can optionally be used with the lipid:nucleic acid complexes of this invention to target specific cells or tissues. In one embodiment, the targeting moiety, such as an antibody or antibody fragment, is attached to a hydrophilic polymer and is combined with the lipid:nucleic acid complex after complex formation. Thus, the use of a targeting moiety in combination with a generic effector lipid:nucleic acid complex provides the ability to conveniently customize the complex for delivery to specific cells and tissues.

Examples of effectors in lipid:nucleic acid complexes include nucleic acids encoding cytotoxins (e.g., diphtheria toxin (DT), Pseudomonas exotoxin A (PE), pertussis toxin (PT), and the pertussis adenylate cyclase (CYA)), antisense nucleic acid, ribozymes, labeled nucleic acids, and nucleic acids encoding tumor suppressor genes such as p53, p110Rb, and p72. These effectors can be specifically targeted to cells such as cancer cells, immune cells (e.g., B and T cells), and other desired cellular targets with a targeting moiety. For example, as described above, many cancers are characterized by overexpression of cell surface markers such as HER2, which is expressed in breast cancer cells, or IL17R, which is expressed in gliomas. Targeting moieties such as anti-HER2 and anti-IL17R antibodies or antibody fragments are used to deliver the lipid:nucleic acid complex to the cell of choice. The effector molecule is thus delivered to the specific cell type, providing a useful and specific therapeutic treatment.

D. Liposome Preparation and Composition

In an other aspect, the present invention relates to a lipid-nucleic acid complex comprising a nucleic acid and a PEP compound of Formula I. As indicated above, the methods of this invention involve complexing a nucleic acid with a compound of Formula I.

In certain aspects, the formulations further comprise a cationic lipid. The term “cationic lipid” refers to any of a number of lipid species which carry a net positive charge at physiological pH. Such lipids include, but are not limited to, DODAC, DOTMA, DDAB, DOTAP, DC-Chol and DMRIE. Additionally, a number of commercial preparations of cationic lipids are available which can be used in the present invention. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA).

The cationic lipid can be used alone, or in combination with a “helper” lipid. Preferred helper lipids are non-ionic or uncharged at physiological pH. Particularly preferred non-ionic lipids include, but are not limited to, cholesterol and DOPE, with cholesterol being most preferred. The molar ratio of cationic lipid to helper can range from 2:1 to about 1:2, more preferably from about 1.5:1 to about 1:1.5 and most preferably is about 1:1.

In addition, the cationic lipids of this invention can be formulated into liposomes. Liposomes are constructed by well known techniques, such as described in Liposome Technology, Vols. 1-3 (G. Gregoriadis, Ed., CRC Press, 1993). Lipids are typically dissolved in chloroform and spread in a thin film over the surface of a tube or flask by rotary evaporation. If liposomes comprised of a mixture of lipids is desired, the individual components are mixed in the original chloroform solution. After the organic solvent has been eliminated, a phase consisting of water optionally containing buffer and/or electrolyte is added and the vessel agitated to suspend the lipid. Optionally, the suspension is then subjected to ultrasound, either in an ultrasonic bath or with a probe sonicator, until the particles are reduced in size and the suspension is of the desired clarity. For transfection, the aqueous phase is typically distilled water and the suspension is sonicated until nearly clear, which requires some minutes depending upon conditions, kind, and quality of the sonicator. Commonly, lipid concentrations are 1 mg/mL of aqueous phase, but could easily be higher or lower by a factor of ten.

The liposomes of the present invention comprise one or more of the cationic lipids of Formula I. Liposomes according to the invention optionally have one or more other amphiphiles. The exact composition of the liposomes will depend on the particular circumstances for which they are to be used. Those of ordinary skill in the art will find it a routine matter to determine a suitable composition. The liposomes of the present invention comprise at least one PEP of the present invention. In a preferred embodiment, the liposomes of the present invention comprise a single type of lipid of Formula I. In another preferred embodiment, the liposomes comprise mixtures of compounds of Formula I. In yet another preferred embodiment, the liposomes of the present invention comprise one or more lipids of Formula I in a mixture with one or more natural or synthetic lipids, e.g., cholesterol or DOPE.

In a preferred embodiment, mostly unilamellar liposomes are produced by the reverse phase evaporation method of Szoka & Papahadjopoulos, Proc. Natl. Acad. Sci. USA, 75: 4194-4198 (1978). Unilamellar vesicles are generally prepared by sonication or extrusion. Sonication is generally performed with a bath-type sonifier, such as a Branson tip sonifier at a controlled temperature as determined by the melting point of the lipid. Extrusion may be carried out by biomembrane extruders, such as the Lipex Biomembrane Extruder. Defined pore size in the extrusion filters may generate unilamellar liposomal vesicles of specific sizes. The liposomes may also be formed by extrusion through an asymmetric ceramic filter, such as a Ceraflow Microfilter, commercially available from the Norton Company, Worcester Mass.

Following liposome preparation, the liposomes that have not been sized during formation may be sized by extrusion to achieve a desired size range and relatively narrow distribution of liposome sizes. A size range of about 0.2-0.4 microns allows the liposome suspension to be sterilized by filtration through a conventional filter, typically a 0.22 micron filter. The filter sterilization method can be carried out on a high throughput basis if the liposomes have been sized down to about 0.2-0.4 microns.

Several techniques are available for sizing liposomes to a desired size. One sizing method is described in U.S. Pat. Nos. 4,529,561 or 4,737,323, herein incorporated by reference. Sonicating a liposome suspension either by bath or probe sonication produces a progressive size reduction down to small unilamellar vesicles less than about 0.05 microns in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10: 421-450 (1981). Average liposome diameter may be reduced by sonication of formed liposomes. Intermittent sonication cycles may be alternated with QELS assessment to guide efficient liposome synthesis.

Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve a gradual reduction in liposome size. For use in the present invention, liposomes having a size of about 0.05 microns to about 0.5 microns. More preferred are liposomes having a size of about 0.05 to 0.2 microns.

E. Nucleic Acid

Nucleic acids of all types may be associated with the transfection complexes of the present invention and subsequently can be transfected. These include DNA, RNA, DNA/RNA hybrids (each of which may be single or double stranded), including oligonucleotides such as antisense oligonucleotides, chimeric DNA-RNA polymers, ribozymes, as well as modified versions of these nucleic acids wherein the modification may be in the base, the sugar moiety, the phosphate linkage, or in any combination thereof.

Further, DNA may be in the form of anti-sense, plasmid DNA, parts of a plasmid DNA, product of a polymerase chain reaction (PCR), vectors (P1, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), anti-sense RNA, ribozymes, chimeric sequences, or derivatives of these groups.

From the foregoing it will be clear to those skilled in the art that the transfection complexes such as for example, lipoplexes, liposomes, and the like of the present invention are useful for both in vitro and in vivo application. The liposomes of the present invention will find use for nearly any in vitro application requiring transfection of nucleic acids into cells. For example, the process of recombinant production of a protein.

The nucleic acids may comprise an essential gene or fragment thereof, in which the target cell or cells is deficient in some manner. This can occur where the gene is lacking or where the gene is mutated resulting in under- or over-expression. The nucleic acids can also comprise antisense oligonucleotides. Such antisense oligonucleotides may be constructed to inhibit expression of a target gene. The foregoing are examples of nucleic acids that may be used with the present invention, and should not be construed to limit the invention in any way. Those skilled in the art will appreciate that other nucleic acids will be suitable for use in the present invention as well.

F. Method for Transfecting

In yet another aspect, this invention relates to a method for transfecting a nucleic acid into a cell. The method involves contacting a cell with a lipid-nucleic acid complex or aggregate comprising a nucleic acid and a compound of Formula I. Liposome-nucleic acid complex/aggregates may be prepared by adding an appropriate amount of nucleic acid to a liposome solution. For transfection, PEP head group to nucleic acid phosphates (N/P) is about 2.5:1 to about 10:1. The amount of DNA can vary considerably, but is normally a few to a few tens of micrograms per standard culture dish of cells. Conditions may vary widely, and it is a routine matter and standard practice to optimize conditions for each type of cell, as suppliers of commercial materials recommend. Optimization involves varying the lipid to DNA ratio as well as the total amount of aggregate.

There is currently some uncertainty regarding the precise way in which nucleic acids and cationic lipids interact. In addition, the structure formed both before and during the transfection process is not definitively known. The present invention, however, is not limited by the particular structural type of complex formed by the liposomes and lipid aggregates of the present invention and the nucleic acids to be transfected. The phrase “liposome-nucleic acid aggregate” means any association of liposome or cationic lipid and nucleic acid that is capable of lipofection.

The lipid-nucleic acid aggregate is added to the cells, in culture medium, and left for some tens of minutes to several hours to perhaps overnight. Usually serum is omitted from the culture medium during this phase of transfection. Subsequently, the medium is replaced with normal, serum-containing medium and the cells are incubated for hours to days or possibly cultured indefinitely.

G. Drug Delivery

In still yet another aspect, this invention relates to a pharmaceutical composition or other drug delivery composition for administering a nucleic acid particle to a cell. This composition includes a lipid-nucleic acid complex comprising a nucleic acid and an amphiphilic cationic lipid of Formula I, and a pharmaceutically acceptable carrier therefor. As used herein, the term “pharmaceutical composition” means any association of a liposome or cationic lipid of Formula I and a nucleic acid and or a mixture of a conventional drug capable of be delivered into cells.

Cationic lipid-assisted drug delivery may be accomplished in the following manner. For drugs that are soluble in organic solvents, such as chloroform, the drug and cationic lipid are mixed in solvents in which both are soluble, and the solvent is then removed under vacuum. The lipid-drug residue is then dispersed in an appropriate aqueous solvent, which, in a preferred embodiment, is sterile physiological saline. The suspension then may optionally be subjected to up to several freeze/thaw cycles. It is then sonicated, either merely to reduce the coarseness of the dispersion or to reduce the particle size to 20-30 nm diameter, depending upon whether large or small particle size is most efficacious in the desired application. For some applications, it may be most effective to generate extruded liposomes by forming the suspension through a filter with pores of 100 nm diameter or smaller. For some applications, inclusion of cholesterol or natural phospholipids in the mixture used to generate the lipid-drug aggregate can be appropriate.

The liposome-drug aggregate may then be delivered in any suitable manner. For drugs that are soluble in aqueous solution and insoluble in organic solvents, the lipid mixture to be used for the lipid dispersion or liposomes is coated on the inside surface of a flask or tube by evaporating the solvent from a solution of the mixture. In general, for this method to be successful, the lipid mixture must be capable of forming vesicles having single or multiple lipid bilayer walls and encapsulating an aqueous core. The aqueous phase containing the dissolved drug, preferably a physiological saline solution, is added to the lipid, agitated to generate a suspension, and then optionally frozen and thawed up to several times.

To generate small liposomes the suspension is subjected to ultrasonic waves for a time necessary to reduce the liposomes to the desired average size. If large liposomes are desired, the suspension is merely agitated by hand or on a vortex mixer until a uniform dispersion is obtained, i.e., until visually observable large particles are absent. If the preparation is to have the drug contained only within the liposomes, then the drug in the aqueous phase is eliminated by dialysis or by passage through a gel-filtration chromatographic column (e.g., agarose) equilibrated with the aqueous phase containing all normal components except the drug. The lipid mixture used can contain cholesterol or natural lipids in addition to the cationic compounds of the present invention. The liposome-drug aggregate may then be delivered in any suitable manner.

H. Disease Treatment

In yet another aspect of the invention comprises novel methods of treating diseases arising from infection by a pathogen or from an endogenous DNA deficiency. These methods comprise administering a liposome-nucleic acid aggregate and/or liposome-drug aggregate solution to a mammal suffering from a pathogenic infection or DNA deficiency. If the disease is the result of infection by a pathogen, the nucleic acid can be an antisense oligonucleotide targeted against an DNA sequence in the pathogen that is essential for development, metabolism, or reproduction of the pathogen. If the disease is a DNA deficiency (i.e., wherein certain endogenous DNA is missing or has been mutated), resulting in under- or over-expression, the nucleic acid maybe the normal DNA sequence.

Several methods of in vivo lipofection have been reported. In the case of whole animals, the lipid-nucleic acid aggregate may be injected into the blood stream, directly into a tissue, into the peritoneum, instilled into the trachea, or converted to an aerosol, which the animal breathes. Zhu, et al., Science 261, 209-211 (1993) describe a single intravenous injection of 100 micrograms of a mixture of DNA and DOTMA:dioleoylphosphatidylethanaolamine that efficiently transfected virtually all tissues. It is also possible to use a catheter to implant liposome-DNA aggregates in a blood vessel wall, which can result in successful transformation of several cell types, including endothelial and vascular smooth muscle cells. Stribling, et al., Proc. Natl. Acad. Sci. USA 89, 11277-11281 (1992), demonstrated that aerosol delivery of a chloramphenicol acetyltransferase (CAT) expression plasmid complexed to cationic liposomes produced high-level, lung-specific CAT gene expression in mice in vivo for at least 21 days. They described the following procedure: Six milligrams of plasmid DNA and 12 μmol of DOTMA/DOPE liposomes were each diluted to 8 mL with water and mixed; equal volumes were then placed into two Acorn I nebulizers (Marquest, Englewood, Colo.); animals were loaded into an Intox small-animal exposure chamber (Albuquerque) and an air flow rate of 4 L/min was used to generate the aerosol (about 90 min were required to aerosolize this volume) the animals were removed from the chamber for 1-2 hours and the procedure was repeated. This protocol is representative of the aerosol delivery method.

The following Examples are presented for illustrative purposes only and are not intended, and should not be construed, to limit the invention in any manner.

### II. EXAMPLES

Chemical. The purity of all compounds was determined to be greater than 95% by thin layer chromatography and ¹H NMR spectroscopy. 4-(N,N-Dimethylamino)-1-butanamine, used in the preparation of polyamines 7 and 10, was synthesized according to a literature procedure (see, Urry et al., J. Am. Chem Soc., 86:2224-2229 (1964)). The transfection reagents TransFast (Promega Corporation, Madison, Wis.) and LipofectAMINE PLUS (Gibco Life Technologies, Gaithersburg, Md.) were commercially obtained and used as directed. Dioleoylphosphatidylethanolamine (DOPE) was purchased from Avanti Polar Lipids, Inc. (Alabaster, Ala.). All other chemicals were obtained from commercial sources and used without further purification. Nuclear magnetic resonance spectroscopy was performed with Varian 300 and 400 MHz instruments in CDCl₃, with residual CHCl₃ as an internal standard for ¹³C NMR and tetramethylsilane as an internal standard for ¹H NMR.

Liposomes. The polyamines and co-lipids were dissolved in chloroform and aliquoted into vials to give the desired concentrations. The solvent was removed by rotary evaporation and trace solvent was removed by placing the sample vial under vacuum for at least 12 hours prior to use. The resultant lipid films were hydrated using nuclease-free water at room temperature, followed by vortex mixing and brief sonication (ca. 1-2 minutes) to obtain turbid liposomal suspensions. LipofectAMINE PLUS (a lipid suspension co-formulated of DOSPA and DOPE at a 3:1 (w/w) ratio) and TransFast (a dehydrated lipid film (0.4 mg) consisting of DMDHP and DOPE) were used as supplied by the manufacturer. Following the manufacturer's instructions, TransFast was frozen overnight, then thawed and vortex mixed prior to using.

Plasmid DNA. Vector pCMV.FOX.Luc-2 contains the firefly luciferase DNA sequence under transcriptional control of the immediate early promoter of human CMV. This vector was prepared as an endotoxin-reduced, supercoiled plasmid using anion exchange resins (Qiagen, Santa Clarita, Calif.). The plasmid vector pNDeGFP contains a jellyfish (Aequorea victoria) green fluorescent protein (GFP) DNA sequence (eGFP vector, Clontech, Palo Alto, Calif.) ligated into the pND plasmid under transcriptional control by the human CMV immediate early promoter.

Ethidium Bromide Displacement Assay. Ethidium bromide (153 μL of a 0.01% solution) was added to 96 μg of pCMV.FOX.Luc-2. This DNA-ethidium bromide solution was diluted to a final volume of 6 mL. Different concentrations of the polyamine liposomes were added to each well of a 96 well microplate, to which 50 μL of the DNA-ethidium bromide solution was added to obtain a final volume of 100 μL. After 5 min. the lipoplexes were treated with 100 μL of water or buffer and gently shaken. The resulting fluorescence at 250 nm excitation and 610 nm emission was measured using a SpectraMax XS microplate fluorometer (Molecular Devices, Oregon).

Particle Size Analysis. A 600 μL lipoplex suspension was made by adding 7.2 μg of pCMV.FOX.Luc-2 to an appropriate volume of the polyamine liposomes to obtain the desired N/P ratio. (The N/P ratio is a mole ratio of amines in the lipid head group to DNA phosphates.) A 500 μL aliquot of this lipoplex suspension was transferred to a 60×5 mm test tube and the particle size was analyzed by dynamic laser light scattering using a NICOMP 380ZLS Particle Sizer (Particle Sizing Systems, Santa Barbara, Calif.). The data were compiled using a NiCOMP algorithm and are reported as intensity/weight distribution.

Cell Culture. CHO cells (ATCC, Rockville, Md.) were cultured in 75 cm² cell culture flasks with HAMS F12 media containing 10% bovine calf serum. NIH 3T3 cells were grown in Dulbecco's modified Eagle's medium with 10% fetal calf serum. The cells were maintained at 37° C. in a 5% CO₂ environment. Twenty-four hours prior to transfection, cells were split into 24 well plates at 60% confluence.

Transfection of CHO and NIH 3T3 Cells. Liposomal suspensions of the polyamines were diluted with Dulbecco's modified essential media (serum free) to obtain the desired concentration. The suspensions were vortex mixed followed by addition of an appropriate quantity of DNA to yield lipoplex preparations at the desired N/P ratio. Commercial formulations were prepared by diluting the stock solutions and complexing with pDNA as suggested by the manufacturer. Immediately prior to transfection, the cells were prepared by aspiration of the growth media. The lipoplex preparation was added to the cells (200 μL, 1 μg DNA/well). After incubation for 2 hours, the lipoplex solution was aspirated and replaced with growth media (500 μL),

Luciferase Assay. Cells were lysed 24 hours after lipoplex administration by adding 200 μL lysis buffer. Luciferase activity was assayed using the Enhanced Luciferase Assay Kit and a Moonlight 2010 luminometer (Pharmingen, San Diego, Calif.). Luciferase activity was measured as Relative Light Units (RLUs) from an aliquot of the cell lysate over a 10 sec. period. A luciferase standard curve was produced using serial dilutions of purified firefly luciferase (Pharmingen, San Diego, Calif.). Using the standard curve, relative light units are converted to μg of luciferase per μL of sample assayed, and expressed as μg luciferase per 60,000 cells. To ensure luminometer consistency, the instrument is calibrated with a luminometer light standard containing ¹⁴C-toluene (Pharmingen, San Diego, Calif.).

Flow Cytometry. Cells transfected with the plasmid vector pNDeGFP were trypsinized 24 h after transfection and resuspended in Dulbecco's phosphate buffered saline (DPBS). The resuspended cells were incubated 30 min. in a solution (1:40 dilution of commercial stock) containing annexin V conjugated to biotin (CalTag, Burlingame, Calif.). After a second DPBS wash, the cells were resuspended 30 min. in a solution (1:400 dilution of commercial stock) of the Tri-color fluorophore conjugated to streptavidin (CalTag, Burlingame, Calif.). After a final DPBS wash, the cells were resuspended in 500 μL DPBS. The cells were then analyzed using a dual channel FACScan (Becton Dickinson, San Jose, Calif.) with a 15 mW, 488 nm argon laser.

Using standard techniques, FACScan gating was set with control cells that were prepared simultaneously with the transfected cells. Viable cells were identified using forward scatter (FSC) and side scatter (SSC). The gates were set to exclude cellular debris. GFP fluorescence was measured with a 530/30 band pass filter on channel FL1. The Tri-color fluorescence was measured with a 675 nm band pass filter on channel FL2. Transfection and mean expression were determined using the FL1 autofluorescence signal from control cells, compared to the transfected cell population.

Actinomycin-D (Sigma, St. Louis, Mo.) was used to treat non-transfected cells 24 hours prior to the assay to induce apoptosis. Immediately prior to flow cytometry analysis, the cells treated with actinomycin-D were treated with annexin V as previously described and used as positive controls for FL2. Non-treated cells were analyzed to establish baseline cytotoxicity and background auto-fluorescence. Each condition was transfected in triplicate and ten thousand cells were counted and simultaneously analyzed for GFP (transfection and expression) and Tri-color (annexin V) fluorescence. Data is reported as cell fractions expressing GFP, Tri-color, or both fluorescent signals.

### Example 1

This Example illustrates that the pentaerythritol structure has an effect on transfection efficacy.

LipofectAMINE PLUS and TransFast were compared in transfection experiments with a polyamine panel providing standard measures to correlate the effect of linker structure with transfection efficacy and cytotoxicity. The structures are set forth below:

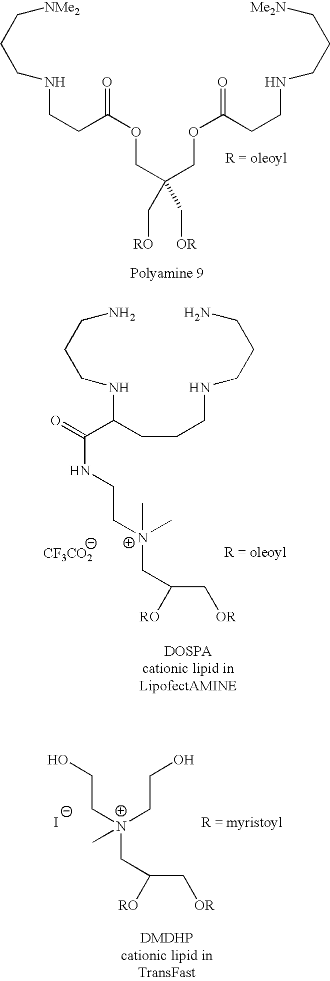

Since these reagents employ different linkers, direct comparison with polyamines 5-10 in transfection experiments provides contrast for evaluating the pentaerythritol linker strategy. In two cell lines, qualitative structural trends from the luciferase experiment were reproduced and thus confirmed using the GFP vector. In addition to the general preference for the diolcoyl hydrophobic domain, the spacing between headgroup nitrogen atoms influences transfection activity. Among compounds with similar hydrophobic domains, polyamines containing C₃-spacers had superior activity in CHO cells and C₂-spacers had superior activity in NIH-3T3 cells. Based on these results, transfection activity correlates to polyamine spacing. Polyamines 8 and 9 exhibited transfection activity which was roughly comparable to LipofectAMINE PLUS and TransFast, but exhibited dramatically lower cytotoxicity.

### Example 2

This Example illustrates that the pentaerythritol structure has an effect on cytotoxicity.

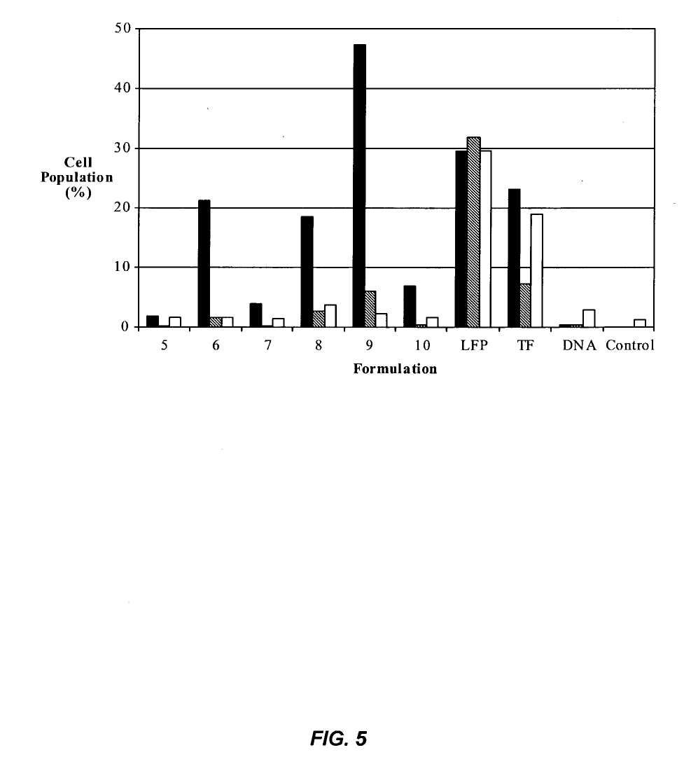

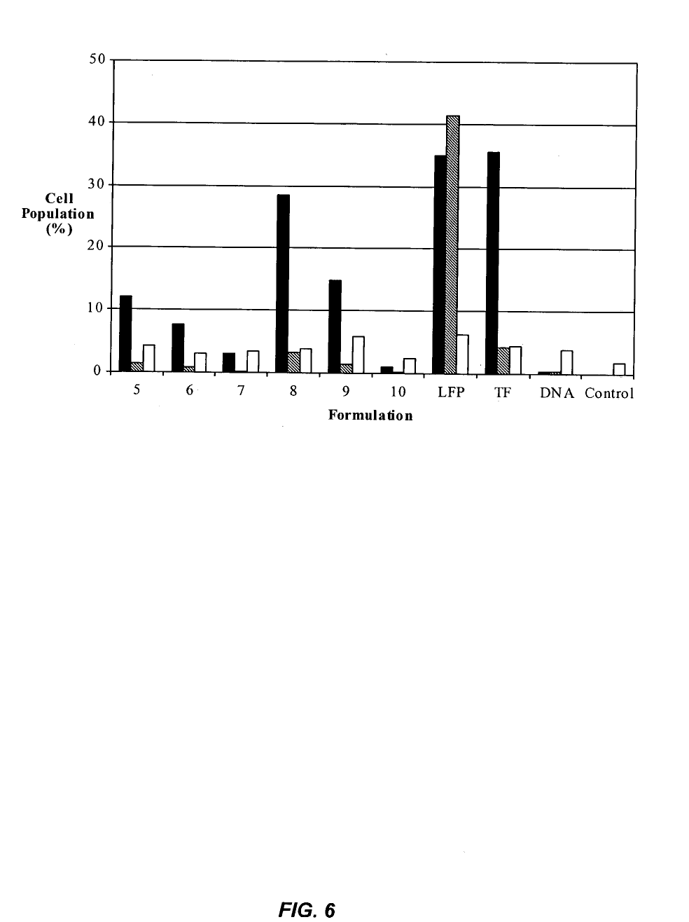

The flow cytometry experiments demonstrated that CHO cells treated with a LipofectAMINE PLUS or TransFast formulation exhibited 38% and 74% cell viability, respectively (FIG. 5). In contrast, CHO cells that were treated with the polyamine formulations demonstrated greater than 90% cell viability. Although the level of gene expression correlates with the level of cytotoxicity for the polyamine formulations, this trend occurs only within this class of compounds. The transfection of CHO cells with compound 9 or LipofectAMINE PLUS produced similar levels of gene expression and dramatically different levels of cytotoxicity. The transfection of NIH 3T3 cells with LipofectAMINE PLUS or TransFast of NIH 3T3 resulted in 53% and 91% cell viability, respectively (FIG. 6). The polyamine formulations resulted in greater than 90% cell viability.

An even more pronounced difference in cytotoxicity between polyamine and commercial lipoplex preparations was noted when analyzing the subset of cells expressing GFP (FIGS. 5 and 6). In CHO cells, polyamine 9 clearly afforded the largest population of cells expressing GFP with extremely low cytotoxicity (FIG. 5, black bars). Although CHO and NIH-3T3 cells treated with LipofectAMINE PLUS also efficiently expressed GFP, analysis of the GFP-positive cell fraction indicated that approximately 50% of these cells were near death in both cell lines. In contrast, cells that were expressing GFP as a result of polyamine-mediated transfection demonstrated only minor (<7%) cytotoxicity.

To further determine the influence of the chemical linker on cytotoxicity, flow cytometry was used to measure cell viability. Using a GFP-annexin V flow cytometric assay, cytotoxicity was simultaneously evaluated in transfected and total cell populations. During apoptosis, phosphatidylserine is translocated to the outside of the plasma membrane, allowing annexin V to bind. Annexin V is an excellent indicator of apoptosis due to its high affinity for phosphatidylserine. Cells undergoing necrosis will also be bound by annexin V due to the disruption of the plasma membrane, as membrane disruption allows annexin V to permeabilize the cell and subsequently bind phoshatidylserine residues. Therefore, the quantification of annexin V-bound cells can be directly correlated with cytotoxicity. The benefit of incorporating a pentaerythritol linker is clearly seen in FIGS. 5 and 6 where cell viability is not compromised by polyamine-mediated transfection. Compounds 5-10 were uniformly non-toxic whereas LipofectAMINE PLUS and, to a lesser extent, TransFast exhibited significantly higher levels of apoptosis and/or cell necrosis. More importantly, when looking at the sub-population of cells expressing GFP, the polyamine-treated cells were more viable in comparison to cells treated with the commercial reagents. This feature is an invaluable characteristic for a gene therapy application that requires repeated administration. As such, pentaerythritol lipids, as a class, are dramatically less cytotoxic, presumably a consequence of their heightened susceptibility to ester hydrolysis. Given that the linker is the only significant difference between the two polyamines (8 and 9) and DOSPA, the flow cytometry results underscore the role of the pentaerythritol linker in ameliorating cytotoxicity associated with lipid-mediated DNA transfection.

### Example 3

This Example illustrates that lipoplex formulation correlates with transfection efficacy.

The polyamine was co-formulated with either cholesterol or DOPE in a 1:1 molar ratio. The lipid mixtures were hydrated and added to plasmid DNA encoding firefly luciferase at polyamine nitrogen/DNA phosphate (N/P) ratios of 2.5:1, 5:1, and 10:1, giving rise to six lipoplex formulations per polyamine compound. Prior to testing these formulations in transfection assays, the particle size of each lipoplex formulation was measured using dynamic laser light scattering. Particle size analysis revealed lipoplex aggregates with diameters principally in the range of 120-240 nm (±11-32 nm).

Formulations containing cholesterol exhibited superior transfection activity compared to the corresponding DOPE formulations. Furthermore, higher N/P ratios were considerably more active than the 2.5:1 formulation. Based on these results, the 1:1 polyamine:cholesterol formulations at the 5:1 amine/DNA phosphate ratio were examined in subsequent cytotoxicity studies. A similar study was conducted using NIH 3T3 cells. As observed with CHO cells, polyamines were more active in NIH 3T3 cells when co-formulated with cholesterol rather than DOPE. In contrast to the CHO cells, an N/P ratio of 10:1 was better than 5:1 in the NIH 3T3 cells.

### Example 4

This Example illustrates a comparison study between the present compounds and other cationic lipids.

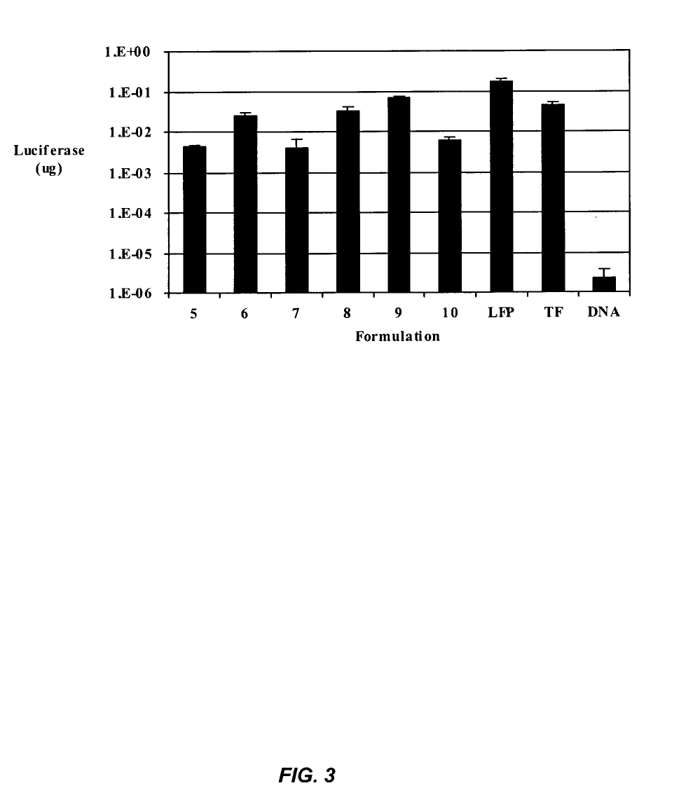

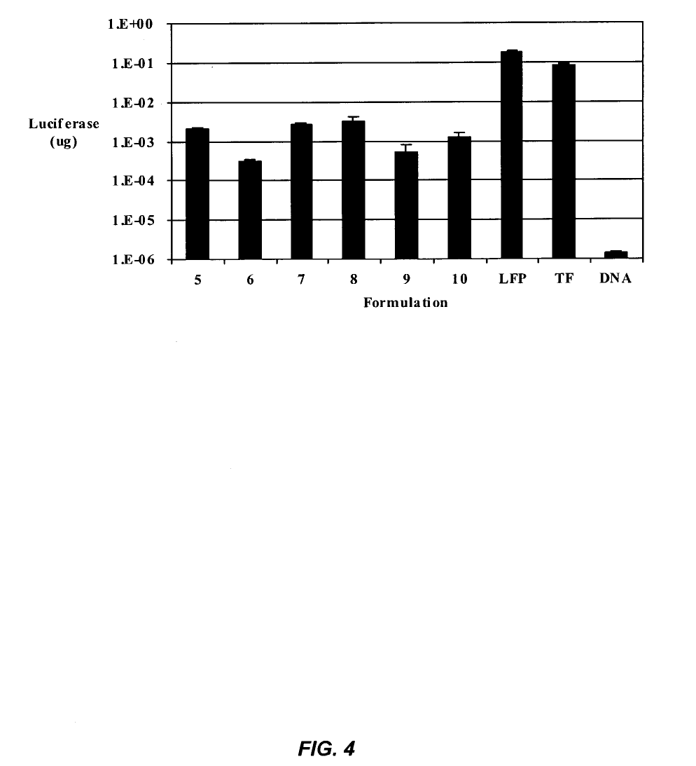

The PEP formulations of the present invention were compared with the commercially available DNA transfection reagents LipofectAMINE PLUS (see, Jessee et al., W. G. Genetic Immunization with Cationic Lipids, Patent No. WO 9427435 (1994)) and TransFast (see, Nantz et al., Polyfunctional Cationic Cytofectins, U.S. Pat. No. 5,869,715 (1999)). Prior to comparisons with the polyamine panel, optimal conditions were determined for transfection of CHO and NIH 3T3 cells using these commercial reagents. Polyamines 5-10 (Scheme 1) and the two commercial reagents were separately formulated with the luciferase plasmid DNA. Analogous lipoplexes were prepared by adding the polyamine formulations to plasmid DNA encoding jellyfish green fluorescent protein. The transfection activities of all formulations were then compared by administration of the luciferase-lipoplexes to CHO cells (FIG. 3) and to NIH 3T3 cells (FIG. 4).

In a second experiment, CHO cells were treated with the lipoplexes containing GFP plasmid. After 24 h, the GFP-treated cells were incubated with the apoptosis marker annexin V and subsequently analyzed using flow cytometry (FIG. 5) (see, Vermes et al., J. Immunol. Meth., 184:39-51 (1995)). By measuring the fluorescence of GFP and the annexin V conjugate, this experiment provides a simultaneous evaluation of the transfection efficiency and cytotoxicity. Except for the different N/P ratio, an identical experiment was conducted in NIH 3T3 cells (FIG. 6).

As shown in FIGS. 3-6, polyamine transfection activity was dependent on the two structural variables, the hydrophobic domain composition and the distribution of amines in the headgroup. In both cell lines, polyamines 8, 9 and 10, which contains the cis-unsaturated oleoyl chains, demonstrated higher levels of both luciferase expression (FIGS. 3,4) and GFP expression (FIGS. 5, 6) when compared to the dimyristoyl analogs 5, 6 and 7 with the same DNA-binding domain. In CHO cells, the polyamines containing propylene spacers between headgroup amines yielded higher levels of gene expression than the corresponding ethylene and butylene analogs. Thus, the dioleoyl derivative with the three carbon spacing, polyamine 9, was the most active analog in this cell line. In NIH 3T3 cells, the transfection activity of the ethylene derivatives, 5 and 8, were superior to the other polyamines. These experiments were repeated and similar trends were noted.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for designing a lipoplex targeted for a specific cell, said method comprising:
providing a lipoplex comprising a nucleic acid and a pentaerythritol polyamine (PEP) compound; and
varying the structural features of said pentaerythritol polyamine (PEP) compound having a hydrophobic domain and a nucleic acid binding domain to impart cell selectivity, thereby designing a lipoplex targeted for said specific cell.

2. The method of claim 1, wherein said cell is a tissue.

3. The method of claim 2, wherein said tissue is a member selected from the group consisting of a tumor, an organ, and bone.

4. The method of claim 3, wherein said tumor is a cancerous tumor.

5. The method of claim 1, wherein said PEP compound has the formula: 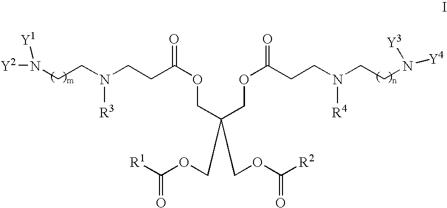
wherein:
R¹ and R² are each members independently selected from the group consisting of optionally substituted C₈-C₂₄ alkyl, optionally substituted -C₈C₂₄ alkenyl, and cholesteryl;
R³ and R⁴ are each members independently selected from the group consisting of hydrogen, and optionally substituted C₁-C₄ alkyl;
Y¹, Y², Y³ and Y⁴ are each members independently selected from the group consisting of hydrogen and optionally substituted C₁-C₆ alkyl;
m and n are integers independently selected from about 1 to about 4, wherein m is not equal to n unless m and n are equal to 3; or a pharmaceutically acceptable salt thereof.

6. The method of claim 5, wherein R¹ and R² are each members independently selected from the group consisting of C₈-C₂₀ alkyl and C₈-C₂₀ alkenyl; and
Y¹, Y², Y³ and Y⁴ are each members independently selected from hydrogen, methyl, ethyl, propyl and butyl.

7. The method of claim 6, wherein R¹ and R² are each members independently selected from the group consisting of C₁₃ alkyl, C₁₇ alkenyl, C₁₁ alkyl, C₇ alkyl and C₁₅ alkyl; and
Y¹, Y², Y³ and Y⁴ are each members independently selected from hydrogen and methyl.

8. The method of claim 7, wherein R¹ and R² are both C₁₇ alkenyl.

9. The method of claim 7, wherein R¹ and R² are both C₁₃ alkyl.

10. The method of claim 5, wherein said pharmaceutically acceptable salt is at least one quaternary nitrogen salt selected from the group consisting of a quaternary ammonium chloride, a quaternary ammonium iodide, a quaternary ammonium fluoride, a quaternary ammonium bromide, a quaternary ammonium oxyanion and a combination thereof.

11. The method of claim 1, wherein said hydrophobic domain is varied by independently changing the chain length of R¹ and R².

12. The method of claim 1, wherein said nucleic acid domain is varied by changing the substituent at Y¹, Y², Y³ and Y⁴.

13. The method of claim 1, wherein the integer of m and n are each independently varied.

14. The method of claim 1, wherein the mole ratio of amines in the PEP head group to nucleic acid phosphates (N/P) is greater than about 2.5:1.

15. The method of claim 1, wherein the mole ratio of amines in the PEP head group to nucleic acid phosphates (N/P) is about 2.5:1 to about 10:1.

16. The method of claim 1, wherein said nucleic acid is plasmid DNA.

17. The method of claim 1, wherein said nucleic acid is antisense RNA or DNA.

18. The method of claim 1, wherein said lipoplex comprises a second lipid.

19. The method of claim 1, wherein said second lipid is a member selected from the group consisting of DOSPA, DOPE, DMDHP, cholesterol, and combinations thereof.

20. A compound having Formula I: 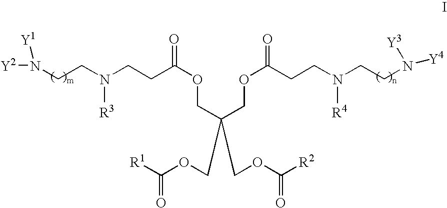
wherein:
R¹ and R² are each members independently selected from the group consisting of optionally substituted C₈-C₂₄ alkyl, optionally substituted C₈-C₂₄ alkenyl, and cholesteryl;
R³ and R⁴ are each members independently selected from the group consisting of hydrogen, and optionally substituted C₁-C₄ alkyl;
Y¹, Y² Y³ and Y⁴ are each members independently selected from the group consisting of hydrogen and optionally substituted C₁-C₆ alkyl;
m and n are integers independently selected from about 1 to about 4, wherein m is not equal to n unless m and n are equal to 3; or a pharmaceutically acceptable salt thereof.

21. The compound of claim 20, wherein R¹ and R² are each members independently selected from the group consisting of C₈-C₂₀ alkyl and C₈-C₂₀ alkenyl; and
Y¹, Y², Y³ and Y⁴ are each members independently selected from hydrogen, methyl, ethyl, propyl and butyl.

22. The compound of claim 20, wherein R¹ and R² are each members independently selected from the group consisting of C₁₃ alkyl, C₁₇ alkenyl, C₁₁ alkyl, C₁₇ alkyl and C₁₅ alkyl; and
Y¹, Y², Y³ and Y⁴ are each members independently selected from hydrogen and methyl.

23. The compound of claim 20, wherein R¹ and R² are both C₁₇ alkenyl.

24. The compound of claim 20, wherein R¹ and R² are both C₁₃ alkyl.

25. The compound of claim 20, wherein said compound has at least one quaternary nitrogen forming a pharmaceutically acceptable salt selected from the group consisting of a quaternary ammonium chloride, a quaternary ammonium iodide, a quaternary ammonium fluoride, a quaternary ammonium bromide, a quaternary ammonium oxyanion and a combination thereof.

26. A transfection complex comprising a nucleic acid and a compound having Formula I: 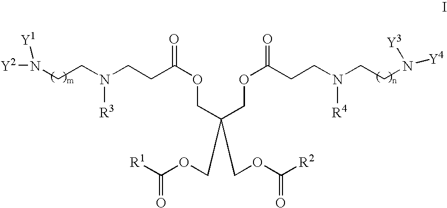
wherein:
R¹ and R² are each members independently selected from the group consisting of optionally substituted C₈-C₂₄ alkyl, optionally substituted C₈-C₂₄ alkenyl, and cholesteryl;
R³ and R⁴ are each members independently selected from the group consisting of hydrogen, and optionally substituted C₁-C₄ alkyl;
Y¹, Y², Y³ and Y⁴ are each members independently selected from the group consisting of hydrogen and optionally substituted C₁-C₆ alkyl;
m and n are integers independently selected from about 1 to about 4, wherein m is not equal to n unless m and n are equal to 4; or a pharmaceutically acceptable salt thereof.

27. The transfection complex of claim 26, wherein R¹ and R² are each members independently selected from the group consisting of C₈-C₂₀ alkyl and C₈-C₂₀ alkenyl; and
Y¹, Y², Y³ and Y⁴ are each members independently selected from hydrogen, methyl, ethyl, propyl and butyl.

28. The transfection complex of claim 26, wherein R¹ and R² are each members independently selected from the group consisting of C₁₃ alkyl, C₁₇ alkenyl, C₁₁ alkyl, C₁₇ alkyl and C₅ alkyl;
Y¹, Y², Y³ and Y⁴ are each members independently selected from hydrogen and methyl.

29. The transfection complex of claim 26, wherein R¹ and R² are both C₁₇ alkenyl.

30. The transfection complex of claim 26, wherein R¹ and R² are both C₁₃ alkyl.

31. The transfection complex of claim 26, wherein said nucleic acid is plasmid DNA.

32. The transfection complex of claim 26, wherein said nucleic acid is antisense RNA or DNA.

33. The transfection complex of claim 26, wherein said compound has at least one quaternary nitrogen forming a pharmaceutically acceptable salt selected from the group consisting of a quaternary ammonium chloride, a quaternary ammonium iodide, a quaternary ammonium fluoride, a quaternary ammonium bromide, a quaternary ammonium oxyanion and a combination thereof.

34. A method for transfecting a nucleic acid into a cell, said method comprising:
contacted a cell with a lipid-nucleic acid complex comprising a compound of Formula I: 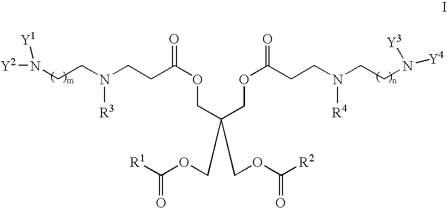
wherein:
R¹ and R² are each members independently selected from the group consisting of optionally substituted C₈-C₂₄ alkyl, optionally substituted C₈-C₂₄ alkenyl, and cholesteryl;
R³ and R⁴ are each members independently selected from the group consisting of hydrogen, and optionally substituted C₁-C₄ alkyl;
Y¹, Y², Y³ and Y⁴ are each members independently selected from the group consisting of hydrogen and optionally substituted C₁-C₆ alkyl;
m and n are integers independently selected from about 1 to about 4, wherein m is not equal to n unless m and n are equal to 4; or a pharmaceutically acceptable salt thereof, thereby transfecting said nucleic acid into a cell.

35. The method of claim 34, wherein R¹ and R are each members independently selected from the group consisting of C₈-C₂₀ alkyl and C₈-C₂₀ alkenyl; and
Y¹, Y², Y³ and Y⁴ are each members independently selected from hydrogen, methyl, ethyl, propyl and butyl.

36. The method of claim 34, wherein R¹ and R² are each members independently selected from the group consisting of C₃ alkyl, C₁₇ alkenyl, C₁₁ alkyl, C₁₇ alkyl and C₁₅ alkyl;
Y¹, Y², Y³ and Y⁴ are each members independently selected from hydrogen and methyl.

37. The method of claim 34, wherein R¹ and R² are both C₁₇ alkenyl.

38. The method of claim 34, wherein R¹ and R² are both C₁₃ alkyl.

39. The method of claim 34, wherein said nucleic acid is plasmid DNA.

40. The method of claim 34, wherein said nucleic acid is antisense RNA or DNA.

41. The method of claim 34, wherein said compound has at least one quaternary nitrogen forming a pharmaceutically acceptable salt selected from the group consisting of a quaternary ammonium chloride, a quaternary ammonium iodide, a quaternary ammonium fluoride, a quaternary ammonium bromide, a quaternary ammonium oxyanion and a combination thereof.
